(19) United States
(12) Patent Application Publication
(10) Pub. No.: US 2010/0087832 A1
(43) Pub. Date: Apr. 8, 2010

(54) INTRAOCULAR LENS INJECTOR

(76) Inventor: William J. Seyboth, Rochester, NY (US)

Correspondence Address: Bausch & Lomb Incorporated, One Bausch & Lomb Place, Rochester, NY, 14604-2701, US

(21) Appl. No.: 12/244,871

(22) Filed: Oct. 3, 2008

(51) Int. Cl. A61F 9/007

(52) U.S. Cl. 606/107

(57) ABSTRACT

An IOL injector, comprising an injector body, a plunger and a plunger collar. The injector body has a lumen wall defining a lumen, a port through which a lens is inserted into a loading bay of the lumen, a stop, and an opening adapted for delivery of an IOL into an eye. The plunger comprising a plunger body, a plunger shaft and a plunger tip. The collar comprises a hole or a channel, the plunger shaft extending therethrough. The plunger shaft is slidably, frictionally engaged with the collar. The plunger and the collar are arranged within the lumen such that an outer surface of the collar can smoothly slide within the lumen wall from a first location where the tip is proximal of an IOL disposed at the loading bay to a second location where the stop interferes with the collar. The collar having an outer dimension that is substantially equal to a dimension of the lumen.

### FIELD OF INVENTION

The present invention relates to intraocular lens (IOL) injectors, and more particularly to IOL injectors providing plunger control.

### BACKGROUND OF THE INVENTION

IOLs are artificial lenses used to replace or supplement the natural crystalline lenses of patients' when their natural lenses are diseased or otherwise impaired. IOLs may be placed in either the posterior chamber or the anterior chamber of an eye. IOLs come in a variety of configurations and materials.

Various instruments and methods for implanting such IOLs in an eye are known. Typically, an incision is made in a patient's cornea and an IOL is inserted into the eye through the incision. In one technique, a surgeon uses surgical forceps to grasp the IOL and insert it through the incision into the eye. While this technique is still practiced today, more and more surgeons are using IOL injectors, which offer advantages such as affording a surgeon more control when inserting an IOL into an eye and permitting insertion of IOLs through smaller incisions. Relatively small incision sizes (e.g., less than about 3 mm) are preferred over relatively large incisions (e.g., about 3.2 to 5+mm) since smaller incisions have been attributed with reduced post-surgical healing time and reduced complications such as induced astigmatism.

In order for an IOL to fit through a small incision, it is typically folded and/or compressed by passing the IOL through a nozzle providing a funnel-shaped portion of the injector lumen and/or providing a compressor drawer. After the IOL exits an opening at the distal end of the nozzle and enters the eye, it assumes its original unfolded/uncompressed shape.

FIG. 1A is a schematic illustration of a conventional IOL injector 10. FIG. 1B is a cut away cross section of a portion of the injector of FIG. 1A. Injector 10 comprises an injector body 20, a nozzle 60, and a plunger 30 that is substantially fully retracted within the injector body (i.e., the plunger is substantially fully withdrawn from the injector body). The plunger comprises a plunger body 32 and a plunger shaft 34. The distance traveled by the plunger between full retraction and full extension (i.e., where the plunger is actuated far enough to fully deliver the IOL into an eye) is called the plunger stroke S. In the illustrated embodiment, the plunger shaft is provided with flexible spring arms 36 that extend into gaps 38 in the injector body at locations along the stroke selected to facilitate achieving an amount of longitudinal movement of the plunger. For example, gaps may occur so that the plunger can be staged for shipping and/or gaps may occur at a location where the plunger has pushed an IOL a selected distance down the lumen so that the IOL is staged for handoff from surgical staff to a surgeon during a surgical procedure.

Stability of the plunger 30 within the injector body is desirable to provide a user with a robust feeling when injecting an IOL, and to help ensure proper engagement of plunger tip 40 with IOL 50.

A plunger may be particularly unstable at points of the stroke near full retraction, where interface I between the plunger and the injector body is relatively small. Plunger stability may be particularly troublesome for molded plastic injectors, but also occurs in other injectors.

### SUMMARY

Aspects of the present invention are directed to an IOL injector, comprising an injector body, a plunger and a collar. The injector body comprises a lumen wall defining a lumen, a port through which a lens is inserted into a loading bay of the lumen, a stop, and an opening adapted for delivery of an IOL into an eye. The plunger comprises a plunger body, a plunger shaft and a plunger tip. The collar comprises a hole or a channel. The plunger shaft extends through the hole or channel. The plunger shaft is slidably, frictionally engaged with the collar. The plunger and the collar are arranged within the lumen such that an outer surface of the collar can smoothly slide within the lumen wall from a first location where the tip is proximal of an IOL disposed at the loading bay to a second location where the stop interferes with the collar, the collar having an outer dimension that is substantially equal to a dimension of the lumen (e.g., the outer dimension is suitable to achieve slidability within the lumen wall while limiting the amount of rotation of the plunger).

In some embodiment, the collar is arranged in the injector body such that it is inaccessible to a user. In some embodiments, the stop is located proximally of the loading bay. The collar may be rigid or flexible. In some embodiments, the injector comprises a compressor coupled to the injector body proximate the port.

Another aspect of the invention is directed to an IOL injector, comprising an injector body, a plunger and a collar. The injector body has a lumen wall defining a lumen, and an opening adapted for delivery of an IOL into an eye. The plunger comprises a plunger body, a plunger shaft and a plunger tip. The collar comprises a hole or a channel. The plunger shaft extends through the hole or change, the plunger shaft being slidably, frictionally engaged with the collar. The plunger and the collar arranged are within the lumen such that an outer surface of the collar can slide within the lumen wall, the collar being arranged in the injector body such that it is inaccessible to a user.

### BRIEF DESCRIPTION OF THE DRAWINGS

Illustrative, non-limiting embodiments of the present invention will be described by way of example with reference to the accompanying drawings, in which the same reference number is used to designate the same or similar components in different figures, and in which:

FIGS. 1A and 1B illustrate a projection view and a cross sectional view of a conventional injector, respectively;

FIG. 2 illustrates a projection view of an example injector comprising an example of an embodiment of a collar according to aspects of the present invention;

FIGS. 3A and 3B are expanded front and rear views, respectively, of the embodiment of a collar shown in FIG. 2; and

FIGS. 4A, 4B and 4C are plan, side and front projection views, respectively, of a second embodiment of a collar according to aspects of the present invention.

### DETAILED DESCRIPTION

IOLs are artificial lenses used to replace or supplement the natural crystalline lenses of patients' when their natural lenses are diseased or otherwise impaired. IOLs may be placed in either the posterior chamber or the anterior chamber of an eye. IOLs come in a variety of configurations and materials.

Various instruments and methods for implanting such IOLs in an eye are known. Typically, an incision is made in a patient's cornea and an IOL is inserted into the eye through the incision. In one technique, a surgeon uses surgical forceps to grasp the IOL and insert it through the incision into the eye. While this technique is still practiced today, more and more surgeons are using IOL injectors, which offer advantages such as affording a surgeon more control when inserting an IOL into an eye and permitting insertion of IOLs through smaller incisions. Relatively small incision sizes (e.g., less than about 3 mm) are preferred over relatively large incisions (e.g., about 3.2 to 5+mm) since smaller incisions have been attributed with reduced post-surgical healing time and reduced complications such as induced astigmatism.

In order for an IOL to fit through a small incision, it is typically folded and/or compressed by passing the IOL through a nozzle providing a funnel-shaped portion of the injector lumen and/or providing a compressor drawer. After the IOL exits an opening at the distal end of the nozzle and enters the eye, it assumes its original unfolded/uncompressed shape.

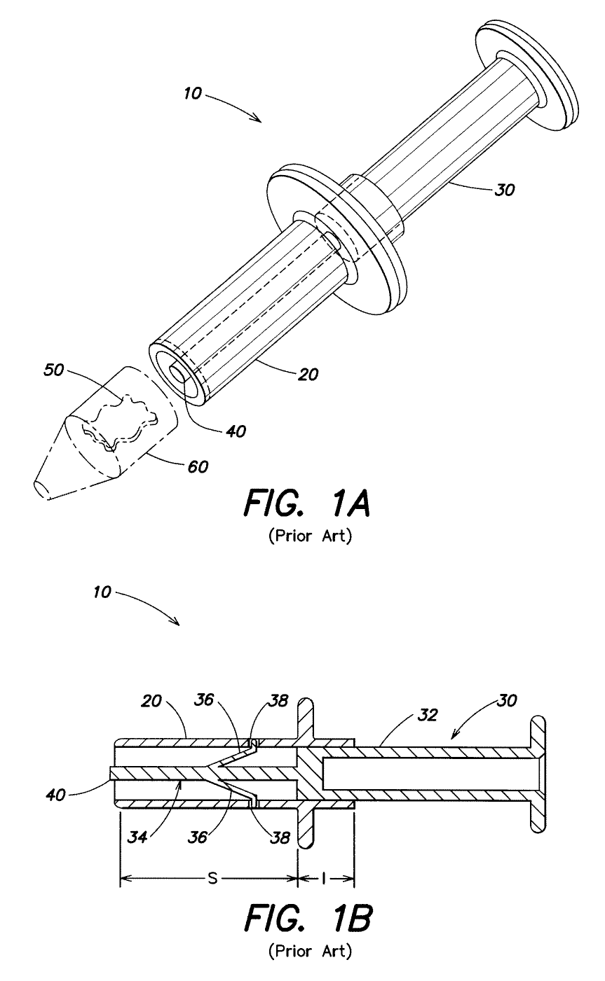

FIG. 1A is a schematic illustration of a conventional IOL injector 10. FIG. 1B is a cut away cross section of a portion of the injector of FIG. 1A. Injector 10 comprises an injector body 20, a nozzle 60, and a plunger 30 that is substantially fully retracted within the injector body (i.e., the plunger is substantially fully withdrawn from the injector body). The plunger comprises a plunger body 32 and a plunger shaft 34. The distance traveled by the plunger between full retraction and full extension (i.e., where the plunger is actuated far enough to fully deliver the IOL into an eye) is called the plunger stroke S. In the illustrated embodiment, the plunger shaft is provided with flexible spring arms 36 that extend into gaps 38 in the injector body at locations along the stroke selected to facilitate achieving an amount of longitudinal movement of the plunger. For example, gaps may occur so that the plunger can be staged for shipping and/or gaps may occur at a location where the plunger has pushed an IOL a selected distance down the lumen so that the IOL is staged for handoff from surgical staff to a surgeon during a surgical procedure.

Stability of the plunger 30 within the injector body is desirable to provide a user with a robust feeling when injecting an IOL, and to help ensure proper engagement of plunger tip 40 with IOL 50.

A plunger may be particularly unstable at points of the stroke near full retraction, where interface I between the plunger and the injector body is relatively small. Plunger stability may be particularly troublesome for molded plastic injectors, but also occurs in other injectors.

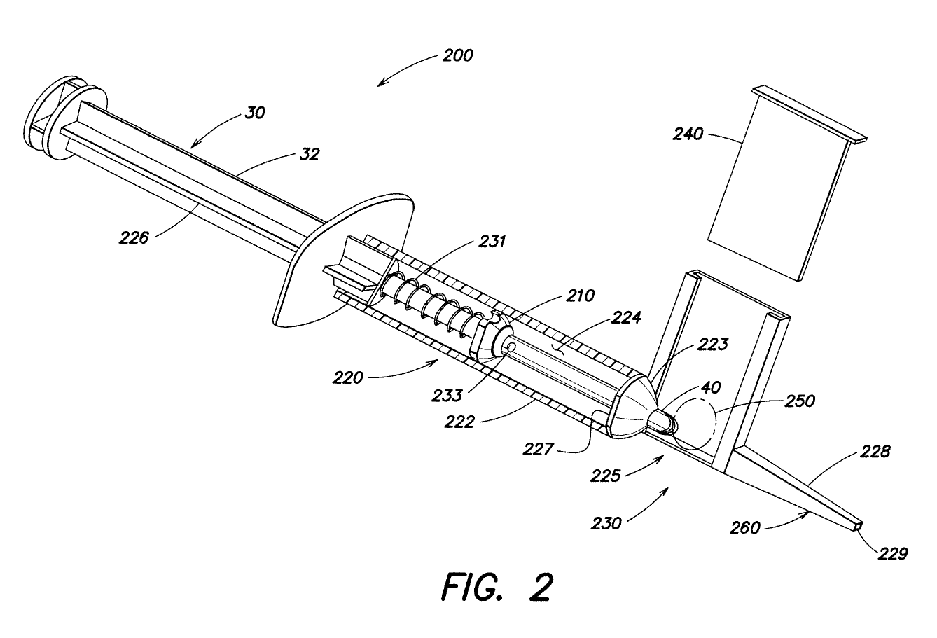

FIG. 2 illustrates a projection view of an example of an IOL injector 200 comprising an example of an embodiment of a collar 210 according to aspects of the present invention. Injector 200 comprises an injector body 220 and a plunger 30, in addition to collar 210.

The injector body has a lumen wall 222 defining a lumen 224, and a port 226 through which a lens is inserted into a loading bay of the lumen. The IOL passes through a distal portion 228 of the lumen (formed in nozzle 260) during injection of an IOL 250. The IOL exits the lumen through an opening 229 configured for delivery of the IOL into an eye. A port provides access to a portion of the lumen referred to as the loading bay 225. In the illustrated embodiment, a compressor 240 is provided that is configured and arranged to compress an IOL at a loading bay, and forms a portion of the lumen between the distal portion and the proximal portion. Further details regarding injectors having a compressor drawer are given in U.S. Pat. No. 5,944,725, titled METHOD AND APPARATUS FOR INSERTING A FLEXIBLE MEMBRANE INTO AN EYE, issued Aug. 31, 1999, to Cicenas, et al, the substance of said patent is hereby incorporated by reference in its entirety. It will be appreciated that, in the illustrated embodiment, at the port, the lumen does not form an enclosed structure when the drawer is open. Although the illustrated embodiment includes a drawer at its port, other embodiments of injectors may have other configurations at their ports. In some embodiments, instead of a drawer, a door (not shown) may be provided over the port to cover the port. In some embodiments, a spring 231 may be included to retract the plunger after insertion of an IOL into an eye.

The plunger comprises a plunger body 32, a plunger shaft 34 and a plunger tip 40. The plunger body interacts with the injector body to maintain the plunger. In some embodiments, the plunger body comprises fins 226 to prevent rotation of the plunger within the injector body. The shaft is a relatively thin portion of the plunger that moves though distal portion of the injector body, and terminates in the plunger tip. The plunger tip may have any suitable configuration for interacting with a lens. For example, a tip may be fork-shaped or a tip may be made of silicone.

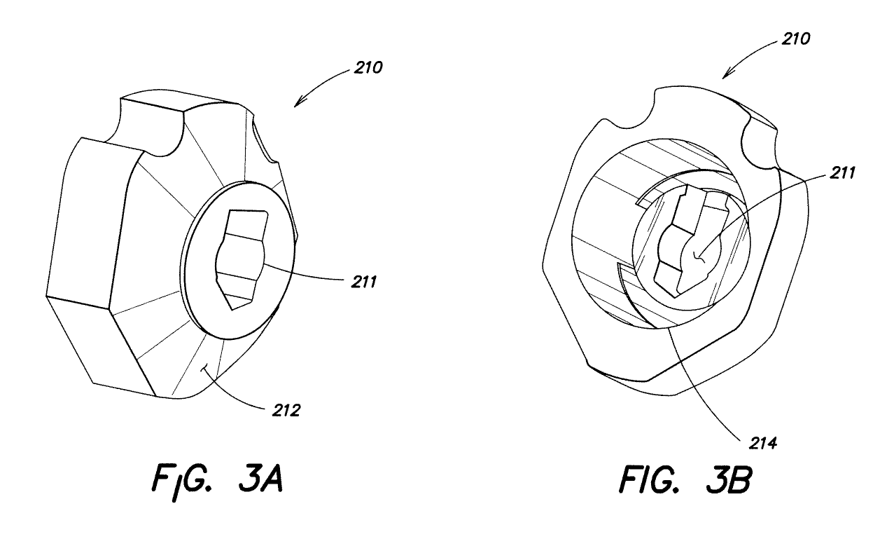

According to aspects of the present invention, the illustrated embodiment of the collar 210 comprises a hole 211 (shown in FIGS. 3A and 3B), through which the plunger shaft extends. As described in greater detail below, the collar interacts with a portion of the lumen wall to facilitate stabilization of the plunger shaft and plunger tip during insertion of an IOL into an eye. Although collar 210 is shown as having a hole, it will be appreciated that a collar may comprise a suitably shaped channel (i.e., a shape that does not fully surround the plunger shaft) which provides a fictional fit to the plunger shaft.

The collar is typically enclosed within the lumen such that, when the plunger and collar are in the injector body (e.g. during movement through the plunger's stroke), the collar cannot be accessed by a user for direct manipulation by the user's fingers. A collar retention feature 233 (e.g., a small bump) may be provided on the plunger shaft to retain the collar. In some embodiments, the cross sectional shape of the shaft and the shape of the hole permit placement of the collar on the plunger in only one orientation.

An objective of some embodiments of injectors according to aspects of the present invention is to provide for plunger shaft and plunger tip stability while maintaining compactness of the injector. Accordingly, the plunger shaft is slidably, frictionally engaged with the collar so that during a first phase of the injection, the plunger slides down the lumen with the collar as a unit. It will be appreciated that slidability of the collar relative to the plunger permits the collar to be positioned on the plunger shaft apart from the plunger body, thereby permitting the plunger body and the collar to limit rotational movement of the plunger within the injector body without limiting the stroke of the plunger. Accordingly, a slidable collar may be particularly suitable for maintaining a relatively compact injector design.

In some embodiments, the collar permits a rotational deviation of the plunger shaft relative to the longitudinal axis of the lumen of less than ±3 degrees when the plunger tip is positioned proximally of an IOL in the loading bay. In other embodiments, the relative movement is less than ±2 degrees when the tip is positioned proximally of an IOL in the loading bay, and in other embodiments, less than ±1 degree. It will be appreciated that precision of the location of the plunger tip, immediately prior to engagement of the lens is desirable for ease of use.

The plunger and the collar are configured and arranged within the lumen such that an outer surface of the collar can smoothly slide within the lumen wall. According to aspects of the present invention, the collar has an outer dimension that is substantially equal to a dimension of the lumen. Such a configuration operates to stabilize the plunger during insertion of a lens into the eye while allowing slidabilty of the plunger and collar unit within the lumen during insertion. It will be appreciated that, for example, smooth sliding is distinct from a configuration in which projections and detents are used to permit the collar to be positioned at discrete locations within the lumen.

In some embodiments, smooth sliding of the plunger and collar as a unit can occur at least from a first location where the tip 40 is proximal of IOL 250 when the IOL is disposed at the loading bay to a more distal location. It will be appreciated that although the collar smoothly slides down lumen in the above manner, the plunger body or shaft may include, for example, a spring arm that engages the injector body (e.g., for staging) as was described in the Background section above. It will also be appreciated that, due to space limitations, at a certain location in the plunger stroke, the collar will interfere with a portion of the injector body, and thereby prevent further translation of the stop down the lumen. The portion of the injector body with which the collar interferes is referred to herein as a stop 227. Accordingly, in the illustrated embodiment, the plunger and collar slide as a unit to a second location, more distally located than the first location, where the stop interferes with the collar. A stop need have no particular configuration. In the illustrated embodiment, the stop is a feature at the end of the proximal portion of the injector body. In the illustrated embodiment, the stop is located proximally of the loading bay.

After interference of the collar and the stop, the stroke of the plunger continues, in a second phase, where the plunger shaft slides relative to the collar (i.e., within the hole or channel of the collar). Such sliding continues until the IOL reaches the opening in the lumen adapted for delivery of an IOL into an eye.

In the embodiment illustrated in FIG. 2, the collar is rigid. For example, to achieve rigidity, the collar is made of polypropylene, and has a suitable minimum thickness. For example, a polypropylene collar having a shape as illustrated in FIG. 2, may have a minimum dimension of at least about 0.5-1 mm. It will be appreciated that the thickness to achieve rigidity is determined by the material, shape of the part and other factors. To achieve slidability of the rigid collar within the lumen while maintaining plunger stability, outer dimensions of the collar are at least about 0.05-0.2 mm less than the corresponding inner dimensions of the lumen. It will be appreciated that the clearance that is appropriate for slidability is determined, for example, by material properties of the collar and lumen wall, and shapes of the collar and lumen wall (which will determine the surface area of the contact region between the collar the lumen wall).

In some embodiments, it is desirable that the collar provide stability without substantially increasing the frictional resistance to the plunger over what would be present in the absence of the collar. A collar may be made of, for example, plastic, elastomer, metal. A surface of a collar may be adapted to form a frictional interface with a lumen wall of the inserter to achieve a particular resistance (i.e., a feel) for a user of the injector.

FIGS. 3A and 3B are expanded front and rear views, respectively, of the embodiment of the collar shown in FIG. 2. A convex distal portion 212 of the collar is configured to match a shape of the end the end portion 223 of the proximal portion of the injector body, and thereby increase the amount of translation of the plunger which may occur. A hollow portion 214 is provided on the rear of the collar to permit the spring to compress therein. As illustrated, the outer surface of the collar may be shaped to form selected regions where the collar makes contact with the inner wall of the lumen. A collar may include features to provide visual cues to assist in assembly and manufacture of the collar.

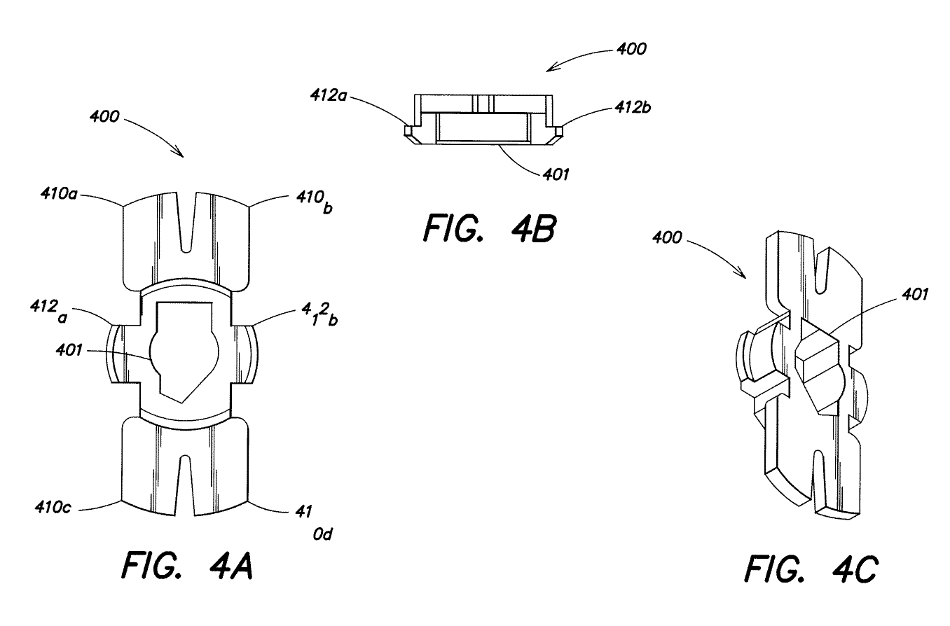

FIGS. 4A, 4B and 4C are plan, side and front projection views, respectively, of a second embodiment 400 of a collar according to aspects of the present invention having a hole 401. An advantage of a collar configured as shown in FIGS. 4A-4C is that it is thin and relatively flexible. Whereas a relatively rigid collar as shown in FIGS. 3A and 3B causes a plunger to reach the end of its stroke when the collar reaches stop 227 (shown in FIG. 2) and the spring 231 (shown in FIG. 2) is fully compressed, a flexible collar 400 permits a further stroke phase during which the collar flexes. It will be appreciated that the terms “flexible” and “rigid” refer characteristics of the collar under forces generated during injection of an IOL.

In the illustrated embodiment, the collar flexes into cone-shaped end 223 (shown in FIG. 2) of the proximal portion of the injector body. It will be appreciated that flexure occurs when translation of the stop (down the lumen) is prevented. Collar 210 comprises four wings that define the outer dimension of the collar. Such a configuration provides four points of maximum radial dimension, while facilitating flexing at the end of the stroke as described above. In some embodiments, for example, the collar is made of polypropylene and the wings of the collar are about 0.5 mm thick.

Additionally, to achieve slidability of the flexible collar illustrated in FIGS. 4A-4C within the lumen 224 while maintaining plunger stability, the outer dimension of the collar can range from about 0.05-0.2 mm less than the corresponding inner dimension of the lumen, to interfering by about 0.02-0.1 mm. Again, it will be appreciated that the dimensions will be determined by the design of the collar (e.g., collar and lumen material, and collar and lumen wall shape). It will be appreciated that, with a flexible collar, it is possible to provide stability without substantially increasing the frictional resistance to the plunger over what would be present in the absence of the collar.

In the illustrated embodiment, collar 210 includes spring retention features 412a and 412b that permit spring (e.g., spring 231) to be attached to the collar. For example, the spring may be wrapped around the retention features.

Having thus described the inventive concepts and a number of exemplary embodiments, it will be apparent to those skilled in the art that the invention may be implemented in various ways, and that modifications and improvements will readily occur to such persons. Thus, the embodiments are not intended to be limiting and presented by way of example only. The invention is limited only as required by the following claims and equivalents thereto.

What is claimed is:

1. An IOL injector, comprising:
an injector body having a lumen wall defining a lumen, a port through which a lens is inserted into a loading bay of the lumen, a stop, and an opening adapted for delivery of an IOL into an eye;
a plunger comprising a plunger body, a plunger shaft and a plunger tip; and
a collar comprising a hole or a channel, the plunger shaft extending therethrough, the plunger shaft being slidably, frictionally engaged with the collar,
the plunger and the collar arranged within the lumen such that an outer surface of the collar can smoothly slide within the lumen wall from a first location where the tip is proximal of an IOL disposed at the loading bay to a second location where the stop interferes with the collar, the collar having an outer dimension that is substantially equal to a dimension of the lumen.

2. The injector of claim 1, wherein the collar is arranged in the injector body such that it is inaccessible to a user.

3. The injector of claim 1, wherein the stop is located proximally of the loading bay.

4. The injector of claim 1, wherein the collar is rigid.

5. The injector of claim 1, wherein the collar is flexible.

6. The injector of claim 1, further comprising a compressor coupled to the injector body proximate the port.

7. The injector of claim 1, wherein the collar has an outer dimension that is substantially equal to a dimension of the lumen, such that the collar permits a rotational deviation of the plunger shaft relative to a longitudinal axis of the lumen of less than ±3 degrees when the plunger tip is positioned proximally of an IOL in the loading bay.

8. The injector of claim 1, wherein the collar has an outer dimension that is substantially equal to a dimension of the lumen, such that the collar permits a rotational deviation of the plunger shaft relative to a longitudinal axis of the lumen of less than ±2 degree.

9. The injector of claim 1, wherein the collar has an outer dimension that is substantially equal to a dimension of the lumen, such that the collar permits a rotational deviation of the plunger shaft relative to a longitudinal axis of the lumen of less than ±1 degree.

10. An IOL injector, comprising:
an injector body having a lumen wall defining a lumen, and an opening adapted for delivery of an IOL into an eye;
a plunger comprising a plunger body, a plunger shaft and a plunger tip; and
a collar comprising a hole or a channel, the plunger shaft extending therethrough, the plunger shaft being slidably, frictionally engaged with the collar,
the plunger and the collar arranged within the lumen such that an outer surface of the collar can slide within the lumen wall, the collar being arranged in the injector body such that it is inaccessible to a user.

11. The injector of claim 10, wherein the collar has an outer dimension that is substantially equal to a dimension of the lumen, such that the collar permits a rotational deviation of the plunger shaft relative to a longitudinal axis of the lumen of less than ±3 degrees when the plunger tip is positioned proximally of an IOL in the loading bay.

12. The injector of claim 10, wherein the collar has an outer dimension that is substantially equal to a dimension of the lumen, such that the collar permits a rotational deviation of the plunger shaft relative to a longitudinal axis of the lumen of less than ±2 degree.

13. The injector of claim 10, wherein the collar has an outer dimension that is substantially equal to a dimension of the lumen, such that the collar permits a rotational deviation of the plunger shaft relative to a longitudinal axis of the lumen of less than ±1 degree.
